SEQUENCE LISTING (3 sequences)

SEQ ID NO: 1 - DNA, 1475 bp, Lactobacillus acidophilus
gggaaagttgcggggtgctatacatgcagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctgggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaagcagtcaggtgttcc

SEQ ID NO: 2 - DNA, 1472 bp, Lactobacillus paracasei
gcaggtggcgggtgctatacatgcagtcgacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaaccctttagggagcgagcgtctaagtggctcacgcct

SEQ ID NO: 3 - DNA, 1492 bp, Lactobacillus plantarum
tatcagtacgtgctataatgcagtcgacgactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctgggggagtacggcccgcaaggctgaaactcaaaggaattgacggggggcccgcacaagcggtgggagcatgtgggtttaattcaaagctacgcgaagaaaccttacccaggttttgacatactaatgcaaattctaaagagattagaacgtttcccttccggggacatgggataccgggtgggtgcatgggttggtcgtcagcttcgtggtcgtgagaatgtttgggtttaagttccccgaaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaacctttagaaccagccgcctaatggcaccaccatgcg

(19) United States
(12) Patent Application Publication
(10) Pub. No.: US 2023/0046756 A1
(43) Pub. Date: Feb. 16, 2023

(54) Lactobacillus Acidophilus KBL409 Strain and Use Thereof

(71) Applicant: KoBioLabs, Inc., Seoul (KR)

(72) Inventors: Tae Wook Nam, Gyeonggi-do (KR); Kiuk Lee, Gyeonggi-do (KR); Seok Cheon Song, Seoul (KR); Seung Hyeok Han, Seoul (KR); Bo Young Nam, Seoul (KR); Ji Min Park, Seoul (KR)

(21) Appl. No.: 17/765,972

(22) PCT Filed: Sep. 29, 2020
(86) PCT No.: PCT/KR2020/013379

§ 371 Date: Oct. 13, 2022

(30) Foreign Application Priority Data
Oct. 2, 2019 (KR) 10-2019-0122531

(51) Int. Cl. C12N 1/20; A23L 33/135; A23K 10/16; A61K 35/747; A61P 13/12

(52) U.S. Cl. CPC C12N 1/20; A23L 33/135; A23K 10/16; A61K 35/747; A61P 13/12; A23V 2200/3204

(57) ABSTRACT

The present invention relates to a Lactobacillus acidophilus KBL409 strain and uses thereof, wherein the Lactobacillus acidophilus KBL409 (deposit number KCTC 13518BP) strain according to the present invention reduces inflammation of the kidney, reduce the concentration of uremic toxins such as blood urea nitrogen, creatinine and p-cresol to protect the kidney, and thus may be usefully utilized for prophylactic and therapeutic applications of renal diseases including improving renal function and chronic renal failure.

### TECHNICAL FIELD

The present invention relates to a Lactobacillus acidophilus KBL409 strain and uses thereof, and more particularly, to a novel probiotic Lactobacillus acidophilus KBL409 strain and a pharmaceutical composition for preventing or treating renal disease, a food composition and an animal feed composition containing at least one selected from the group consisting of the strain, a culture, a lysate and an extract thereof.

### BACKGROUND ART

Chronic renal disease (CKD) is a disease that has emerged as a global health problem, and it has been reported in the United States that 13% of the entire study population shows chronic renal failure at a frequency of one of ten in the world. Chronic renal failure is difficult to expect a reversible recovery easily, and dialysis or transplantation must be prepared in the state of end-stage renal failure if the renal function gradually decreases and enters 5 stages of chronic renal failure.

The treatment of renal failure known so far is focused only on the treatment of complications resulting from loss of renal function, and there is no drug that may fundamentally restore renal failure. In recent decades, several drugs have been tried to prevent the progression to renal failure, but only the degree of drug blocking the Renin-angiotensin system (RAS) only relieves renal failure to some extent, and there is no obvious drug clinically useful for restoring it to date.

As kidney failure progresses, various complications occur as uremic toxin accumulates in the body. These uremic toxins are considered to be the cause of major complications, such as inducing inflammatory responses and oxidative stress in the body, and promoting aging of blood vessels (Cachofeiro V et al. Oxidative stress and inflammation, a link between chronic kidney disease and cardiovascular disease. Kidney Int Suppl 2008:S4-9; Wu J et al. The role of oxidative stress and inflammation in cardiovascular aging. Biomed Res Int 2014; 2014:615312). Urine substances include p-cresyl sulfate (PCS), indoxyl sulfate (IS), and trimethylamine-N-oxide (TMAO), and it has been reported that the level of PCS, IS, TMAO in serum and urine is higher than normal for patients with chronic renal failure (Ramezani A et al., Role of the Gut Microbiome in Uremia: A Potential Therapeutic Target. Am J Kidney Dis. 2016 67:483-498). These are secondary metabolites metabolised in the liver after uptake of p-cresol, indole, Trimethylamine (TMA), which is a metabolite produced by the degradation of tyrosine (or phenylalanine), tryptophan and choline in the intestine of the food by the intestinal microbiota, via epithelial cells (see FIG. 25 below).

The most important cause of accumulation of these uremic toxins in the body is a decrease in renal excretion. However, a significant portion of uremic toxins are nitrogen waste products generated in the intestine after food is orally absorbed, and this is greatly affected by the intestinal environment and microorganisms. For this reason, the concept of ‘enteric dialysis’ was introduced, and the intestinal mucosa acts as a semi-permeable membrane to excrete some wastes into the intestine. In fact, drugs developed to inhibit the absorption of uremic toxins that occur in the intestine are being used in clinical practice, and drugs such as phosphorus-binding agents and potassium lowering inhibitors are drugs that induce specific substances such as phosphorus or potassium from being not absorbed in the intestine. In addition, AST-120 (Kremezin®, Kureha-Chemical Co., Tokyo, Japan) is an oral adsorbent composed of carbon microspheres, and it is a drug that induces excretion through feces by adsorbing indole-based substances which is a representative uremic toxin occurring in the intestine. However, most of these drugs have side effects of the digestive system such as indigestion, nausea, vomiting, and constipation, so the compliance with the drug is lower than that of other general drugs. Therefore, there is still no effective method to remove uremia other than dialysis or transplantation.

Probiotics refer to microorganisms and products produced by the microorganisms having antibacterial and enzymatic activities that help balance the gut microbiome. In addition, probiotics are defined as live bacteria in the form of single or consortium that are supplied to humans or animals in the form of dried cells or fermentation products to improve the gut microbiome. The characteristics that probiotics should possess are that the human intestine is the habitat, have non-pathogenic and non-toxic characteristics, and must survive while going to the intestine. Furthermore, it must maintain survival rate and activity before consumption in the delivered food, be sensitive to antibiotics used to prevent infection, and must not harbor antibiotic-resistant plasmids. In addition, it must have resistance to acids, enzymes, and bile in the intestinal environment. Recently, probiotics have been spotlighted as a major therapeutic substance that may replace existing compounds-based therapeutic agent as various effects of improving health functions have been reported.

Through various recent studies, the population of healthy intestinal microorganisms plays an important role in regulation of nutrition and metabolism, immune response, and an imbalance in intestinal microorganisms has been reported to be involved in various diseases such as obesity, type 2 diabetes, inflammatory bowel disease, cardiovascular complications, etc. In the case of chronic renal failure, the growth of strains that are not seen in normal persons in the duodenum and jejunum of patients is also observed, and in particular hyperproliferation of aerobic strains is seen, some reports have reported that strains such as Enterobacteria or Enterococci increase by more than 100 fold in hemodialysis patients (Simenhoff M L et al. Biomodulation of the toxic and nutritional effects of small bowel bacterial overgrowth in end-stage kidney disease using freeze-dried Lactobacillus acidophilus. Miner Electrolyte Metab 1996; 22:92-96; Hida M et al., Inhibition of the accumulation of uremic toxins in the blood and their precursors in the feces after oral administration of Lebenin, a lactic acid bacteria preparation, to uremic patients undergoing hemodialysis. Nephron 1996; 74:349-355). There is also a report in patients with chronic renal failure that Lactobacillus or Bifidobacteria strains known to balance the proper balance of the intestinal microbial environment, produce short chain fatty acids to inhibit infection of yeast, fungi and harmful bacteria, and secrete various metabolites are reduced, ultimately resulting in intestinal imbalance (Koppe L et al., Probiotics and chronic kidney disease. Kidney Int 2015; 88:958-966; Ramezani A & Raj D S. The gut microbiome, kidney disease, and targeted interventions. J Am Soc Nephrol 2014; 25:657-670; Ranganathan N et al. Pilot study of probiotic dietary supplementation for promoting healthy renal function in patients with chronic kidney disease. Adv Ther 2010; 27:634-647).

Therefore, attempts have been recently reported to reduce complications due to uremic disease and improve renal function by converting the unbalanced intestinal bacterial environment into a healthy environment in patients with chronic renal failure. In particular, when probiotics comprising Lactobacillus and Bifidobacteria strains were administered for 16 weeks experimentally in a ⅚ nephrectomy model using Sprague Dawley rats, the nitrogen level in the blood decreased and the effect of improving renal function using probiotics was expected (Ranganathan N et al. Probiotic amelioration of azotemia in ⅚th nephrectomized Sprague-Dawley rats. ScientificWorldJournal 2005; 5:652-660). Patients have also shown positive results in clinical studies, and it has been shown in 1996 that they lower blood urea nitrogen level (BUN), which is an indicator of renal failure, when probiotics are administered in patients with chronic renal failure since they reported blood dimethylamine (DMA) and Nitrosodimethylamine by orally administering Lactobacillus strains to patients with terminal renal failure who are undergoing 8 dialysis (Ranganathan N et al. Pilot study of probiotic dietary supplementation for promoting healthy renal function in patients with chronic kidney disease. Adv Ther 2010; 27:634-647; Ranganathan N et al. Probiotic dietary supplementation in patients with stage 3 and 4 chronic kidney disease: a 6-month pilot scale trial in Canada. Curr Med Res Opin 2009; 25:1919-1930). In a randomized, double-blind study in which patients with chronic renal failure were first progressed, preparation of enteric coated capsules in which probiotics and prebiotics were mixed and administration to a patient at stage 4 of chronic renal insufficiency was observed to significantly reduce the p-cresol blood level, which is an important intestinal development uremic, while not reducing indoxyl sulfate levels in the blood of the whole patient, but significantly reducing in patients not exposed to antibiotics. In addition, it was reported that there is a significant increase in Bifidobacteria strains in the intestinal bacteria analysis, which may improve the intestinal environment (Rossi M et al. Synbiotics Easing Renal Failure by Improving Gut Microbiology (SYNERGY): A Randomized Trial. Clin J Am Soc Nephrol 2016; 11:223-231).

### SUMMARY OF INVENTION

### Technical Problem

Accordingly, the present inventors have completed the present invention by confirming that a novel Lactobacillus acidophilus strain has excellent effects in terms of suppressing renal inflammation, reducing uremic toxins, reducing proteinuria, restoring function of renal mitochondria and suppressing renal fibrosis, and is useful for improving renal function and treating or preventing renal disease for the treatment of renal disease, and as a result, it has been made to study probiotics for kidney diseases including chronic renal failure which is not a satisfactory treatment in the past.

### Solution to Problem

It is an object of the present invention to provide a novel Lactobacillus acidophilus strain and various uses thereof, which show excellent effects in improving renal function, or preventing and treating renal disease.

In order to achieve the above object, the present invention provides a Lactobacillus acidophilus KBL409 strain with deposit number KCTC 13518BP.

The present invention also provides the pharmaceutical composition for preventing or treating renal disease, containing at least one selected from the group consisting of the strain, a culture of the strain, a lysate of the strain, and an extract of the strain.

The pharmaceutical composition according to the present invention may prevent or treat renal disease through decrease in renal inflammation, decrease in blood concentration of uremic toxin, decrease in proteinuria, restoration of renal mitochondrial function and/or an inhibition of renal fibrosis.

In the present invention, the uremic toxin may comprise blood urea nitrogen, blood creatinine and/or blood p-cresol.

In the present invention, the renal disease may be selected from, but not limited to, the group consisting of uremia, chronic renal failure, acute renal failure, subacute renal failure, renal fibrosis, glomerulonephritis, pyelonephritis, interstitial nephritis, proteinuria, diabetic nephropathy, hypertensive nephropathy, malignant neurosis, lupus nephritis, thrombotic microangiopathy, transplant rejection, glomerulopathy, renal hypertrophy, renal hyperplasia, contrast agent induced nephropathy, toxin induced kidney injury, oxygen free-radical mediated nephropathy, polycystic renal disease and nephritis.

The present invention also provides a food composition containing one or more selected from a group consisting of the strain, the culture of the strain, the lysate of the strain and the extract of the strain.

The present invention also provides an animal feed composition containing one or more selected from a group consisting of the strain, the culture of the strain, the lysate of the strain and the extract of the strain.

The pharmaceutical composition of the present invention may be administered in combination with an additional probiotic strain capable of enhancing the renal function improving effect of the Lactobacillus acidophilus strain (KBL409) of the present invention, such as Lactobacillus paracasei and/or Lactobacillus plantarum. Preferably, said additional probiotic strain comprises Lactobacillus paracasei KBL382 (deposit number KCTC13509BP) strain and/or Lactobacillus plantarum KBL396 (deposit number KCTC13278BP).

The present invention also provides the method for preventing or treating renal disease, comprising administering to an individual in need thereof at least one selected from the group consisting of the strain, a culture of the strain, a lysate of the strain, and an extract of the strain.

The present invention also provides the use of a composition for preparing a medicament for preventing or treating renal disease, comprising at least one selected from the group consisting of the strain, the culture of the strain, the lysate of the strain and the extract of the strain.

### BRIEF DESCRIPTION OF DRAWINGS

FIG. 1 is a result of comparing p-cresol degrading ability of 67 kinds of strains of Lactobacillus and Lactococcus strains.

FIG. 2 is a result of comparing the p-cresol degrading ability of 33 kinds of Bifidobacterium strains.

FIG. 3 is a result of comparing the p-cresol degrading ability of 10 kinds of Lactobacillus candidate strains showing high p-cresol degrading ability.

FIG. 4 is a result showing the p-cresol degrading ability according to the treatment time of the Lactobacillus acidophilus KBL409 strain.

FIG. 5 is a result confirming the expression levels of IL-2, IL-4, IL-13 and IL-17A of PBMCs when Lactobacillus acidophilus KBL409 strain is treated.

FIG. 6 is a result confirming the expression levels of IFN-7 of PBMCs when Lactobacillus acidophilus KBL409 strain is treated.

FIG. 7 is a result confirming the expression levels of IL-10 of PBMCs when Lactobacillus acidophilus KBL409 strain is treated.

FIG. 8 is a schematic diagram of an experiment for identifying the renal protective effect of Lactobacillus acidophilus KBL409 strain in an animal model of chronic renal failure.

FIG. 9 is a result showing the change in renal function by administration of adenine and Lactobacillus acidophilus KBL409 strain.

FIG. 10 is a result showing the change in albuminuria by administration of adenine and Lactobacillus acidophilus KBL409 strain.

FIG. 11 is a result showing the change in kidney morphology and renal fibrosis by administration of adenine and Lactobacillus acidophilus KBL409 strain: (A) control group, (B) KBL409 administration group, (C) adenine feed administration group, (D) adenine feed and KBL409 administration group.

FIG. 12 is a result showing the change in the mRNA expression of Procol1a and Acta2 by administration of the Lactobacillus acidophilus KBL409 strain upon induction of chronic renal failure.

FIG. 13 is a result showing the change of the protein index related to kidney induced by chronic renal failure fibrosis when Lactobacillus acidophilus KBL409 strain is administered.

FIG. 14 is a result showing the change of macrophage-related indices in the kidney when Lactobacillus acidophilus KBL409 strain is administered.

FIG. 15 is a result of confirming the change of macrophages in chronic renal failure-induced renal tissue by administration of Lactobacillus acidophilus KBL409 strain through immunohistochemical examination: (A) F4/80 staining, (B) CD68 staining.

FIG. 16 is a result showing the mRNA expression changes of Tlr4, Asc, Nlrp3, IL-18 in the kidney induced by chronic renal failure by the administration of Lactobacillus acidophilus KBL409 strain.

FIG. 17 is a result confirming the change in NRLP3 activity in chronic renal failure-induced renal tissue by administration of the Lactobacillus acidophilus KBL409 strain through immunohistochemical examination.

FIG. 18 is a result confirming the morphological change of mitochondria in the kidney induced by chronic renal failure by administration of Lactobacillus acidophilus KBL409 strain through transmission electron microscopy.

FIG. 19 is a result showing the change of the systemic inflammatory response in the chronic renal failure induction model when Lactobacillus acidophilus KBL409 strain is administered.

FIG. 20 is a result showing the change in the concentration of the blood p-cresol according to the administration of the Lactobacillus acidophilus KBL409 strain: (A) Comparison with E. coli (B) Comparison between Lactobacillus acidophilus strains.

FIG. 21 is a result showing the change in the concentration of the blood TMAO according to the administration of the Lactobacillus acidophilus KBL409 strain:

FIG. 22 is a result showing the change in the mRNA expression of Nlrp3 and Pre-IL18 in the kidney induced by chronic renal failure by Lactobacillus acidophilus KBL409 strain alone administration, KBL409 and KBL382 strain combined administration, KBL409 and KBL396 strain combined administration.

FIG. 23 is a result showing the change in the mRNA expression of Ppargc1a, Tfam and Mfn1 in the kidney induced by chronic renal failure by Lactobacillus acidophilus KBL409 strain alone administration, KBL409 and KBL382 strain combined administration, KBL409 and KBL396 strain combined administration.

FIG. 24 is a result showing the change in the mRNA expression of Fn and Procol1 in the kidney and changes in the Bax/Bcl2 ratio in the kidney induced by chronic renal failure by Lactobacillus acidophilus KBL409 strain alone administration, KBL409 and KBL382 strain combined administration, KBL409 and KBL396 strain combined administration.

FIG. 25 is a schematic diagram illustrating a pathway through which tyrosine, tryptophan, and choline contained in food are metabolized to uremic toxin through the intestinal microbiota and the liver.

### DESCRIPTION OF EMBODIMENTS

Unless defined otherwise, all technical and scientific terms used herein have the same meaning as commonly understood by one of ordinary skill in the art to which this invention belongs. In general, the nomenclature used herein is those well known and commonly used in the art.

In the present invention, a Lactobacillus acidophilus KBL409 (accession number KCTC 13518BP) strain having an excellent effect of improving renal function was selected by confirming the uremic effect of a human-derived microorganism, and the 16S rDNA of the strain was analyzed to confirm that the strain is a novel strain which has not been known in the prior art.

Accordingly, the present invention relates to a Lactobacillus acidophilus KBL409 (deposit number KCTC 13518BP) strain, which is a novel probiotic from one aspect, wherein the strain is characterized as comprising a 16s rDNA sequence represented by SEQ ID NO: 1 below.

<SEQ ID NO: 1> 16s rDNA sequence of
Lactobacillus acidophilus KBL409
(deposit number KCTC 13518BP) strain
GGGAAAGTTGCGGGGTGCTATACATGCAGTCGAGCGAGCTGAACCAACA
GATTCACTTCGGTGATGACGTTGGGAACGCGAGCGGCGGATGGGTGAGT
AACACGTGGGGAACCTGCCCCATAGTCTGGGATACCACTTGGAAACAGG
TGCTAATACCGGATAAGAAAGCAGATCGCATGATCAGCTTATAAAAGGC
GGCGTAAGCTGTCGCTATGGGATGGCCCCGCGGTGCATTAGCTAGTTGG
TAGGGTAACGGCCTACCAAGGCAATGATGCATAGCCGAGTTGAGAGACT
GATCGGCCACATTGGGACTGAGACACGGCCCAAACTCCTACGGGAGGCA
GCAGTAGGGAATCTTCCACAATGGACGAAAGTCTGATGGAGCAACGCCG
CGTGAGTGAAGAAGGTTTTCGGATCGTAAAGCTCTGTTGTTGGTGAAGA
AGGATAGAGGTAGTAACTGGCCTTTATTTGACGGTAATCAACCAGAAAG
TCACGGCTAACTACGTGCCAGCAGCCGCGGTAATACGTAGGTGGCAAGC
GTTGTCCGGATTTATTGGGCGTAAAGCGAGCGCAGGCGGAAGAATAAGT
CTGATGTGAAAGCCCTCGGCTTAACCGAGGAACTGCATCGGAAACTGTT
TTTCTTGAGTGCAGAAGAGGAGAGTGGAACTCCATGTGTAGCGGTGGAA
TGCGTAGATATATGGAAGAACACCAGTGGCGAAGGCGGCTCTCTGGTCT
GCAACTGACGCTGAGGCTCGAAAGCATGGGTAGCGAACAGGATTAGATA
CCCTGGTAGTCCATGCCGTAAACGATGAGTGCTAAGTGTTGGGAGGTTT
CCGCCTCTCAGTGCTGCAGCTAACGCATTAAGCACTCCGCCTGGGGGAG
TACGACCGCAAGGTTGAAACTCAAAGGAATTGACGGGGGCCCGCACAAG
CGGTGGAGCATGTGGTTTAATTCGAAGCAACGCGAAGAACCTTACCAGG
TCTTGACATCTAGTGCAATCCGTAGAGATACGGAGTTCCCTTCGGGGAC
ACTAAGACAGGTGGTGCATGGCTGTCGTCAGCTCGTGTCGTGAGATGTT
GGGTTAAGTCCCGCAACGAGCGCAACCCTTGTCATTAGTTGCCAGCATT
AAGTTGGGCACTCTAATGAGACTGCCGGTGACAAACCGGAGGAAGGTGG
GGATGACGTCAAGTCATCATGCCCCTTATGACCTGGGCTACACACGTGC
TACAATGGACAGTACAACGAGGAGCAAGCCTGCGAAGGCAAGCGAATCT
CTTAAAGCTGTTCTCAGTTCGGACTGCAGTCTGCAACTCGACTGCACGA
AGCTGGAATCGCTAGTAATCGCGGATCAGCACGCCGCGGTGAATACGTT
CCCGGGCCTTGTACACACCGCCCGTCACACCATGGGAGTCTGCAATGCC
CAAAGCCGGTGGCCTAACCTTCGGGAAGGAGCCGTCTAAGCAGTCAGGT
GTTCC

Therefore, in the present invention, efficacy experiments on the strain were carried out, and as a result, it was confirmed that the strain not only reduces renal inflammation and is excellent in the degradation effect of uremic toxins causing chronic renal failure, but also exhibits the effect of reducing proteinuria, restoring function of renal mitochondria and inhibiting renal fibrosis, and thus exhibits an excellent effect for enhancing renal function to treating or preventing renal disease.

Specifically, the Lactobacillus acidophilus KBL409 strain of the present invention was shown to significantly reduce the concentration of uremic toxins such as blood urea nitrogen, blood creatinine and/or blood p-cresol, and proteinuria in an animal model of chronic renal failure.

In addition, the Lactobacillus acidophilus KBL409 strain of the present invention significantly reduced the expression levels of Procol1a and Acta2 mRNA, which are indicators of renal fibrosis, and Collagen1, Fibronectin, α-SMA and Vimentin, and it was confirmed that histopathological examination also alleviated the renal tubular dilation, the flatness of renal tubular cells, the expansion and substrate accumulation of tubular interstitium, and the increase in renal fibrosis characteristic of renal failure.

Furthermore, the Lactobacillus acidophilus KBL409 strain of the present invention was found to reduce systemic inflammatory responses in a chronic renal failure model by not only inhibiting infiltration of macrophages in the tubular interstitium and expression of inflammasomes in the kidney, but also restoring mitochondrial dysfunction occurring in chronic renal insufficiency and inhibiting IL-6 and TNF-α.

Therefore, in another aspect, the present invention relates to a pharmaceutical composition for the treatment or prevention of a disease, the composition containing at least one pharmaceutically effective amount selected from the group consisting of the fungus of Lactobacillus acidophilus KBL409 strain, the culture of the strain, the lysate of the strain and the extract of the strain.

The pharmaceutical composition of the present invention may be provided as a composition in which fungus of viable bacteria, dried strains, cultures of strains, lysates of strains, or combinations thereof are combined with a pharmaceutically acceptable carrier or medium. The carriers or media employed may comprise solvents, dispersants, coatings, absorption enhancers, controlled release agents (i.e., sustained release agents), and one or more inert excipients (for example, starch, polyols, granules, microfine cellulose, microcrystalline cellulose, diluents, lubricants, binders, disintegrants, etc.), etc. If desired, tablet formulations of the disclosed compositions may be coated by standard aqueous or non-aqueous techniques. Examples of pharmaceutically acceptable carriers and excipients and such additional ingredients include, but are not limited to, binders, fillers, disintegrants, lubricants, antimicrobial agents and coating agents.

The compositions of the present invention may be formulated using methods known in the art to provide rapid, sustained or delayed release of the active ingredient after administration to a mammal. Formulations may be in the form of powders, granules, tablets, emulsions, syrups, aerosols, soft or hard gelatin capsules, sterile injectable solutions, or sterile powders. In addition, the composition for preventing or treating renal disease according to the present invention may be administered through several routes including oral, transdermal, subcutaneous, intravenous or intramuscular, and the dosage of the active ingredient may be appropriately selected to various factors such as the route of administration, age, sex, and weight of the patient, and the severity of the patient, and the composition for preventing or treating renal disease according to the present invention may be administered in combination with a known agent having an effect of improving renal function or preventing or treating renal disease, such as renin-angiotensin system blockers.

The pharmaceutical composition of the present invention may be provided as an enteric-coated enteric preparation, in particular, as a unit dosage form for oral use. As used herein, “enteric coating” comprises all known pharmaceutically acceptable coatings that are not degraded by gastric acid to maintain the coating, but are sufficiently degraded in the small intestine so that the active ingredient may be released into the small intestine. The “enteric coating” of the present invention refers to a coating that maintained as it is for at least 2 hours when it is brought into contact with artificial gastric juice, such as an HCl solution of pH 1, at 36° C. to 38° C., and preferably thereafter, disintegrates within 30 minutes in artificial intestinal juice such as a KH₂PO₄ buffer solution of pH 6.8.

The enteric coating of the present invention is coated in an amount of about 16 to 30, preferably 16 to 20 or not more than 25 mg per core. When the thickness of the enteric coating of the present invention is 5 to 100 m, preferably 20 to 80 m, satisfactory results are obtained as an enteric coating. The material of the enteric coating is appropriately selected from known polymeric materials. Suitable polymeric materials are described in a number of known publications (L. Lachman et al., The Theory and Practice of Industrial Pharmacy, 3rd ed., 1986, pp. 365-H. Sucker et al., Pharmazeutische Technologie, Thieme, 1991, pp. 355-359; Hagers). Handbuchder pharmazeutischen Praxis, 4th ed., Vol. 7, pp. 739-742, and 766-778, (Springer Verlag, 1971); and Remington's Pharmaceutical Sciences, 13th ed., pp. 1689-1691 (Mack Publ., Co., 1970))), which may comprise cellulose ester derivatives, cellulose ethers, methyl acrylate copolymers of acrylic resins and copolymers of maleic acid and phthalic acid derivatives.

The enteric coating of the present invention may be prepared using a conventional enteric coating method in which an enteric coating solution is sprayed onto the core. Suitable solvents used in the enteric coating process comprise alcohols such as ethanol, ketones such as acetone, and halogenated hydrocarbon solvents such as dichloromethane (CH₂Cl₂), and a mixed solvent of these solvents may be used. An emollient such as di-n-butylphthalate or triacetin is added to the coating solution in a ratio of 1 to about 0.05 to about 0.3 (coating material to emollient). It is appropriate to continuously perform the spraying process, and it is possible to adjust the spraying amount in consideration of the conditions of the coating. The spraying pressure may be variously adjusted, and in general, satisfactory results are obtained with a spraying pressure of about 1 to about 1.5 bar.

In the present invention, “renal disease” refers to all conditions in which the kidneys do not normally perform excretion, regulation, metabolism and endocrine functions, and in which overall the function is diminished or aberrant, and includes all chronic kidney diseases, including all diseases which are not apparent and which have a decreased substrate change and glomerular filtration function. Decreased function due to kidney injury results in enlargement of the kidneys and related structures, atrophy of the kidneys, changes in body fluid volume, electrolyte imbalance, metabolic acidosis, gas exchange disorder, impaired anti-infective function, and accumulation of uremic toxins.

In the present invention, the renal disease refers to a disease that may be ameliorated, treated or prevented through decrease in renal inflammation, blood urea nitrogen, reduction of blood concentration of uremic toxins such as blood urea nitrogen, creatinine or p-cresol, decrease in proteinuria, restoration of renal mitochondrial function and/or an inhibition of renal fibrosis. Specifically, the renal disease includes but is not limited to uremia, chronic renal failure, acute renal failure, subacute renal failure, renal fibrosis, glomerulonephritis, pyelonephritis, interstitial nephritis, proteinuria, diabetic nephropathy, hypertensive nephropathy, malignant neurosis, lupus nephritis, thrombotic microangiopathy, transplant rejection, glomerulopathy, renal hypertrophy, renal hyperplasia, contrast agent induced nephropathy, toxin induced kidney injury, oxygen free-radical mediated nephropathy, polycystic renal disease and nephritis.

In the present invention, the term ‘treatment’, unless otherwise stated, means reversing, alleviating, or inhibiting the progression of a disease or disorder to which the term applies, or one or more symptoms of the disease or disorder.

Also, in the present invention, the term ‘prevention’ relates to averting, delaying, impeding, or hindering a disease to diminish.

The composition for preventing or treating renal disease according to the present invention may comprise a pharmaceutically effective amount of Lactobacillus acidophilus KBL409 strain alone or together with one or more pharmaceutically acceptable carriers, excipients or diluents.

In the present invention, the term “effective amount (or pharmaceutically effective amount)” means an amount that is very sufficient to deliver a desired effect but sufficiently small enough to prevent serious side effects within the scope of medical judgment. The amount of microorganisms administered into the body by the composition of the present invention may be appropriately adjusted in consideration of the route of administration and the subject of administration.

The composition of the present invention may be administered to the subject at least once a day. Unit dose means physically separated units suitable for unit dosage for human subjects and other mammals, each unit comprising an appropriate pharmaceutical carrier and Lactobacillus acidophilus KBL409 of the present invention showing therapeutic effect contains a predetermined amount of the strain. The dosage unit for oral administration to an adult patient preferably contains 0.001 g or more of the microorganism of the present invention, and the oral dosage of the composition of the present invention is 0.001 to 10 g, preferably 0.01 to 5 g once. As an example, the pharmaceutically effective amount of the Lactobacillus acidophilus KBL409 strain of the present invention is 0.01 to 10 g/day, and may be administered at a dosage of 1×10⁸ to 1×10¹⁰ CFU/day. However, the dosage will vary depending on the severity of the patient's disease and the microorganism and auxiliary active ingredient used together. In addition, the total daily dosage may be divided into several times and administered continuously as needed. Accordingly, the above dosage ranges do not limit the scope of the present invention in any way.

In another aspect, the present invention relates to a food composition containing one or more selected from a group consisting of Lactobacillus acidophilus KBL409 (deposit number KCTC 13518BP) strain, the fungus of strain, the culture of the strain, the lysate of the strain and the extract of the strain.

The food composition may be a food composition used for improvement in renal function, preferably a health functional food. In addition, such improvement in renal function may be achieved due to a decrease in renal inflammation, a decrease in blood concentration of uremic toxin, a decrease in proteinuria, a restoration of renal mitochondrial function and/or a decrease in renal fibrosis.

The food composition may be easily used as a food effective in improvement in renal function, for example, a main raw material, a supplementary raw material, a food additive, a health functional food or a functional beverage of food, but is not limited thereto.

The food composition means a natural product or processed product containing one or more nutrients, and preferably means a state that may be eaten directly through a certain amount of processing process, and in a general sense, food, which includes all food additives, health functional foods, and functional beverages.

Foods to which the food composition according to the present invention may be added include, for example, various foods, beverages, gum, tea, vitamin complexes, functional foods, etc. In addition, in the present invention, foods include, but are not limited to special nutritional foods (for example, milk formulas, infant food, etc.), processed meat products, fish meat products, tofu, jelly, noodles (for example, ramen, noodles, etc.), breads, health supplements Foods, seasonings (for example, soy sauce, soybean paste, red pepper paste, mixed paste, etc.), sauces, confectionery (for example, snacks), candies, chocolates, gums, ice cream, dairy products (for example, fermented milk, cheese, etc.), other processed foods, kimchi, pickled foods (various kimchi, pickles, etc.), beverages (for example, fruit drinks, vegetable beverages, soy milk, fermented beverages, etc.), natural seasonings (for example, ramen soup, etc.). The food, beverage or food additive may be prepared by a conventional manufacturing method.

The term “health food” refers to a food group in which a food product is imparted with added value so as to exert the function or express the function of the food product for a specific purpose by using a physical, biochemical, biotechnological technique or the like, and food products which have been designed and processed so that they sufficiently express an in vivo regulating function relating to regulation of the in vivo defensive rhythm, prevention of disease, recovery and the like which the food composition has. The functional food may comprise a food supplement additive that is food-related and may further comprise suitable carriers, excipients and diluents commonly used in the manufacture of nutraceuticals.

In the present invention, the functional beverage is a generic term for drinking to resolve brownness or taste, and there is no particular limitation on other ingredients other than comprising the above composition for improving renal function as an essential ingredient in the indicated ratio, and it may contain various flavors or natural carbohydrates and the like as additional ingredients such as conventional beverages.

Furthermore, in addition to those described above, food containing the food composition of the present invention includes various nutrients, vitamins, minerals (electrolytes), flavoring agents such as synthetic and natural flavoring agents, coloring agents and fillers (cheese, chocolate, etc.), pectic acid and its salts, alginic acid and its salts, organic acids, protective colloidal thickeners, pH adjusters, stabilizers, preservatives, glycerin, alcohols, carbonation agents used in carbonated beverages, etc., wherein the components are independently or may be used in combination.

In the food containing the food composition of the present invention, the amount of the composition according to the present invention may comprise 0.001% to 100% by weight of the total food weight, preferably 1% to 99% by weight, and in the case of a beverage, it may be comprised in a ratio of 0.001 g to 10 g, preferably 0.01 g to 1 g, based on 100 mL. However, in the case of long-term intake for health and hygiene or for health control, it may be not more than the above range, and the active ingredient may be used in an amount of not less than the range since there is no problem in terms of safety, and therefore, it is not limited to this range.

The food composition of the present invention may be prepared by adding the Lactobacillus acidophilus KBL409 strain independently or to an acceptable carrier, or in the form of a composition suitable for human or animal ingestion. That is, it may be added to foods that do not contain other probiotic bacteria and foods that already contain some probiotic bacteria. For example, in preparing the food of the present invention, other microorganisms usable with the strains of the invention are those which are suitable for ingestion by humans or animals and which have probiotic activity capable of inhibiting pathogenic harmful bacteria upon ingestion or improving the balance of microorganisms in the mammalian intestinal tract, without particular limitation. Examples of such probiotic microorganisms include yeast comprising Saccharomyces, Candida, Pichia and Torulopsis, fungus such as Aspergillus, Rhizopus, Mucor, Penicillium and bacteria belonging to Lactobacillus, Bifidobacterium, Leuconostoc, Lactococcus, Bacillus, Streptococcus, Propionibacterium, Enterococcus, Pediococcus genus. Specific examples of suitable probiotic microorganisms include Saccharomyces cerevisiae, Bacillus coagulans, Bacillus lichenformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei, Lactobacillus curvatus, Lactobacillus delbruckii, Lactobacillus johnsonii, Lactobacillus farciminus, Lactobacillus gasseri, Lactobacillus helveticus, Lactobacillus rhamnosus, Lactobacillus reuteri, Lactobacillus sakei, Lactococcus lactis, Pediococcus acidilactici etc. Preferably, the effect may be further enhanced by further comprising a probiotic microorganism having an excellent effect of improving renal function while having excellent probiotic activity in the food composition of the present invention. Examples of carriers that may be used in the food composition of the present invention may be extenders, high fiber additives, encapsulating agents, lipids, etc, and examples of such carriers are well known in the art. The Lactobacillus acidophilus KBL409 strain of the present invention may be a freeze-dried or encapsulated form, or a culture suspension or dry powder form.

The composition of the present invention may also be provided in the form of an additive for animal feed containing the strain or a animal feed composition containing the same.

The additive for animal feed of the present invention may be in the form of a dry or liquid formulation, and may further comprise other non-pathogenic microorganisms in addition to the Lactobacillus acidophilus KBL409 strain. Microorganisms that may be added include, for example, Bacillus subtilis which may produce proteolytic enzymes, lipolytic enzymes and sugar converting enzymes, lactose having physiological activity and organics degrading ability under anaerobic conditions such as the stomach of cattle. Lactobacillus strain, filamentous fungus such as Aspergillus oryzae showing the effect of increasing the weight of livestock, increasing the acid flow rate of milk and increasing the digestive absorption rate of feed (Slyter, L. L. J. Animal Sci.1976, 43. 910-926) and yeasts such as Saccharomyces cerevisiae (Johnson, D. E et al. J. Anim. Sci.,1983, 56, 735-739; Williams, P. E. V. et al,1990, 211), etc.

The additive for animal feed of the present invention may further comprise one or more enzyme preparations in addition to the Lactobacillus acidophilus KBL409 strain. The added enzyme preparation may be in both a dry or liquid state, and as the enzyme preparation, a lipolytic enzyme such as lipase, a phytase which decomposes phytic acid to form phosphate and inositol phosphate, an amylase which hydrolyzes α-1,4-glycosidic bonds contained in starch, glycogen, etc, a phosphatase which is an enzyme hydrolyzing organophosphate ester, a carboxymethylcellulase which decomposes cellulose, a xylase which decomposes xylose, a maltase which hydrolyze maltose into two molecules of glucose, a convertase which hydrolyses saccharose to form a glucose-fructose mixture, etc. may be used.

In using the Lactobacillus acidophilus KBL409 strain of the present invention as an additive for animal feed, as raw materials for feed, peanuts including various cereals and soybean protein, peas, sugar beets, pulp, grain by-products, animal interior flour, fish flour, and the like may be used, and these raw materials may be unprocessed or processed without limitation. The processing process is, but not necessarily limited to, for example, a process in which a feed material is charged and compressed under pressure to a constant discharge port, and in the case of a protein, it is preferable to use extrusion molding in which the utilization is increased due to denaturation. Extrusion has advantages such as denaturing proteins and destroying anti-enzyme factors through a heat treatment process. In addition, in the case of soy protein, the nutritional value of the soy protein may be increased by enhancing the digestibility of the protein through extrusion molding, inactivating anti-nutrients such as trypsin inhibitor, which is one of the protease inhibitors present in the soy, and increasing the protease-induced digestibility improvement.

On the other hand, the pharmaceutical, food or animal feed composition of the present invention may further comprise an additional probiotic strain capable of enhancing the renal function improving effect of the KBL409 strain, or the composition may be co-administered simultaneously or sequentially with a separate composition comprising the additional probiotic strains.

The additional probiotic strain capable of enhancing the renal function improvement effect of the KBL409 strain preferably includes Lactobacillus paracasei and Lactobacillus plantarum, more preferably, the additional probiotic strain may be Lactobacillus paracasei KBL382 (deposit number KCTC13509BP) strain having the 16s rDNA sequence of SEQ ID NO: 2 and/or Lactobacillus plantarum KBL396 (deposit number KCTC13278BP) having the 16s rDNA sequence of SEQ ID NO: 3 below.

<SEQ ID NO: 2> 16s rDNA sequence of
Lactobacillus paracasei KBL382
(deposit number KCTC13509BP) strain
GCAGGTGGCGGGTGCTATACATCCAGTCGACGAGTTCTCGTTGATGATC
GGTGCTTGCACCGAGATTCAACATGGAACGAGTGGCGGACGGGTGAGTA
ACACGTGGGTAACCTGCCCTTAAGTGGGGGATAACATTTGGAAACAGAT
GCTAATACCGCATAGATCCAAGAACCGCATGGTTCTTGGCTGAAAGATG
GCGTAAGCTATCGCTTTTGGATGGACCCGCGGCGTATTAGCTAGTTGGT
GAGGTAATGGCTCACCAAGGCGATGATACGTAGCCGAACTGAGAGGTTG
ATCGGCCACATTGGGACTGAGACACGGCCCAAACTCCTACGGGAGGCAG
CAGTAGGGAATCTTCCACAATGGACGCAAGTCTGATGGAGCAACGCCGC
GTGAGTGAAGAAGGCTTTCGGGTCGTAAAACTCTGTTGTTGGAGAAGAA
TGGTCGGCAGAGTAACTGTTGTCGGCGTGACGGTATCCAACCAGAAAGC
CACGGCTAACTACGTGCCAGCAGCCGCGGTAATACGTAGGTGGCAAGCG
TTATCCGGATTTATTGGGCGTAAAGCGAGCGCAGGCGGTTTTTTAAGTC
TGATGTGAAAGCCCTCGGCTTAACCGAGGAAGCGCATCGGAAACTGGGA
AACTTGAGTGCAGAAGAGGACAGTGGAACTCCATGTGTAGCGGTGAAAT
GCGTAGATATATGGAAGAACACCAGTGGCGAAGGCGGCTGTCTGGTCTG
TAACTGACGCTGAGGCTCGAAAGCATGGGTAGCGAACAGGATTAGATAC
CCTGGTAGTCCATGCCGTAAACGATGAATGCTAGGTGTTGGAGGGTTTC
CGCCCTTCAGTGCCGCAGCTAACGCATTAAGCATTCCGCCTGGGGAGTA
CGACCGCAAGGTTGAAACTCAAAGGAATTGACGGGGGCCCGCACAAGCG
GTGGAGCATGTGGTTTAATTCGAAGCAACGCGAAGAACCTTACCAGGTC
TTGACATCTTTTGATCACCTGAGAGATCAGGTTTCCCCTTCGGGGGCAA
AATGACAGGTGGTGCATGGTTGTCGTCAGCTCGTGTCGTGAGATGTTGG
GTTAAGTCCCGCAACGAGCGCAACCCTTATGACTAGTTGCCAGCATTTA
GTTGGGCACTCTAGTAAGACTGCCGGTGACAAACCGGAGGAAGGTGGGG
GGATGACGTCAAATCATCATGCCCCTTATGACCTGGGCTACACACGTGC
TACAATGGATGGTACAACGAGTTGCGAGACCGCGAGGTCAAGCTAATCT
CTTAAAGCCATTCTCAGTTCGGACTGTAGGCTGCAACTCGCCTACACGA
AGTCGGAATCGCTAGTAATCGCGGATCAGCACGCCGCGGTGAATACGTT
CCCGGGCCTTGTACACACCGCCCGTCACACCATGAGAGTTTGTAACACC
CGAAGCCGGTGCGTAACCCTTTAGGGAGCGAGCGTCTAAGTGGCTCACG
CCT
<SEQ ID NO: 3> 16s rDNA sequence of
Lactobbacillus plantarum KBL396
(deposit number KCTC13278BP) strain
TATCAGTACGTGCTATAATGCAGTCGACGACTCTGGTATTGATTGGTGC
TTGCATCATGATTTACATTTGAGTGAGTCGGCGAACTGGTGAGTAACAC
GTGGGAAACTGCCCAGAAGCGGGGGATAACACCTGGAAACAGATGCTAA
TACCGCATAACAACTTGGACCGCATGGTCCGAGCTTGAAAGATGGCTTC
GGCTATCACTTTTGGATGGTCCCGCGGCGTATTAGCTAGATGGTGGGGT
AACGGCTCACCATGGCAATGATACGTAGCCGACCTGAGAGGGTAATCGG
CCACATTGGGACTGAGACACGGCCCAAACTCCTACGGGAGGCAGCAGTA
GGGAATCTTCCACAATGGACGAAAGTCTGATGGAGCAACGCCGCGTGAG
TGAAGAAGGGTTTCGGCTCGTAAAACTCTGTTGTTAAAGAAGAACATAT
CTGAGAGTAACTGTTCAGGTATTGACGGTATTTAACCAGAAAGCCACGG
CTAACTACGTGCCAGCAGCCGCGGTAATACGTAGGTGGCAAGCGTTGTC
CGGATTTATTGGGCGTAAAGCGAGCGCAGGCGGTTTTTTAAGTCTGATG
TGAAAGCCTTCGGCTCAACCGAAGAAGTGCATCGGAAACTGGGAAACTT
GAGTGCAGAAGAGGACAGTGGAACTCCATGTGTAGCGGTGAAATGCGTA
GATATATGGAAGAACACCAGTGGCGAAGGCGGCTGTCTGGTCTGTAACT
GACGCTGAGGCTCGAAAGTATGGGTAGCAAACAGGATTAGATACCCTGG
TAGTCCATACCGTAAACGATGAATGCTAAGTGTTGGAGGGTTTCCGCCC
TTCAGTGCTGCAGCTAACGCATTAAGCATTCCGCCTGGGGGAGTACGGC
CCGCAAGGCTGAAACTCAAAGGAATTGACGGGGGGCCCGCACAAGCGGT
GGGAGCATGTGGGTTTAATTCAAAGCTACGCGAAGAAACCTTACCCAGG
TTTTGACATACTAATGCAAATTCTAAAGAGATTAGAACGTTTCCCTTCC
GGGGACATGGGATACCGGGTGGGTGCATGGGTTGGTCGTCAGCTTCGTG
GTCGTGAGAATGTTTGGGTTTAAGTTCCCCGAAACGAGCGCAACCCTTA
TTATCAGTTGCCAGCATTAAGTTGGGCACTCTGGTGAGACTGCCGGTGA
CAAACCGGAGGAAGGTGGGGATGACGTCAAATCATCATGCCCCTTATGA
CCTGGGCTACACACCTGCTACAATGGATGGTACAACGAGTTGCGAACTC
GCGAGAGTAAGCTAATCTCTTAAAGCCATTCTCAGTTCGGATTGTAGGC
TGCAACTCGCCTACATGAAGTCGGAATCGCTAGTAATCGCGGATCAGCA
TGCCGCGGTGAATACGTTCCCGGGCCTTGTACACCGCCCGTCACACCAT
GAGAGTTTGTAACACCCAAAGTCGGTGGGGTAACCTTTAGAACCAGCCG
CCTAATGGCACCACCATGCG

The Lactobacillus paracasei KBL382 and Lactobacillus plantarum KBL396 may be administered once to several times a day at a dosage of 0.001 to 10 g/day, preferably 0.01 to 5 g/day, respectively.

From the point of view that one or more probiotic strains are administered as a mixture in a single composition, or one or more probiotic strains are administered separately in different compositions, any suitable ration of strains may be used as long as the synergistic probiotic effect of the strains remains useful. These ratios may be readily determined by a person skilled in the art. For example, two strains (such as KBL409:KBL382) in a 1:10, 1:5, 1:1, 5:1, or 10:1 ratio or any ratio between these limits, such as 1:1 may be used.

In another aspect, the present invention provides the use of the strain or composition for use in the prevention or treatment of renal disease, and the use of the strain or composition for the preparation of the therapeutic agent.

In another aspect, the present invention provides a method for preventing or treating the disease, comprising administering a pharmaceutically effective amount of the strain or composition to a subject in need of the prevention or treatment of renal disease.

Since the pharmaceutical composition and administration method used in the method for preventing or treating the disease have been described above, descriptions of common contents between the two are omitted in order to avoid excessive complexity of the present specification.

On the other hand, the subject to which the composition for the prevention or treatment of the disease may be administered includes all animals including humans. For example, it may be an animal such as a dog, cat, or mouse.

Hereinafter, the present invention will be described in more detail through examples. These examples are only for illustrating the present invention, and it will be apparent to those of ordinary skill in the art that the scope of the present invention is not to be construed as being limited by these examples.

### Example 1. Selection of Lactic Acid Bacteria Having p-Cresol Degrading Ability

The precursors of p-cresol sulfate (PCS), indole sulfate (IS), and trimethylamine N-oxide (TMAO), which are the main renal uremic toxins, are p-cresol, indole, trimethylamine (TMA), respectively, and trimethylamine, and are waste products of phenylalanine (or tyrosine), tryptophan, and choline by intestinal microorganisms. The present invention focused on the identification of microorganisms capable of metabolizing/decomposing precursors of renal uremic toxins, and the inventors of the present invention sought to select a probiotic strain exhibiting the effect of treating renal disease through uremic toxin degradation by selecting a p-cresol resolution as the main selection object. To this end, the p-cresol degrading ability of a total of 67 human-derived Lactobacillus and Lactococcus strains, and a total of 33 Bifidobacterium strains was evaluated. All strains were resistant to low pH and bile salts, so they were viable in the gastrointestinal tract for the duration of the course. The p-cresol degrading ability was confirmed by measuring the concentration of the residual p-cresol by gas chromatography after culturing the strains in MRS medium containing p-cresol.

1-1. Culture of Strains and Preparation of Sample

The strains used in this experiment were cultured in MRS medium containing 200 μM of p-cresol for 24 h. Add 2.5 μL of concentrated sulfuric acid to 50 μL of each sample, heat at 90° C. for 30 min, add 2.5 μL of the prepared internal standard (0.2 mg/mL 2,6-dimethylphenol, Sigma-Aldrich), and add 50 μL of ethyl acetate as an analysis solvent and mixed with vortex for 1 minute. After centrifugation at 15,000 rpm for 2 minutes, the supernatant was placed in a GC vial (with 250 μL glass insert).

1-2. Selection of Strains by Measuring the Concentration of p-Cresol

Gas chromatography was used to determine the amount of p-cresol in the culture supernatant. The analysis equipment was GC-EI-MS (Agilent 5985) at a flow rate of 1.3 mL/min, and the analysis was carried out by increasing the oven temperature from the initial 75° C. to 150° C. (rate 20° C.) and 250° C. (rate 25° C.) (Post run 75° C. for 3 min). A DB-5 capillary column 30 m×0.25 mm, df=0.25 (Agilent) was used as the column.

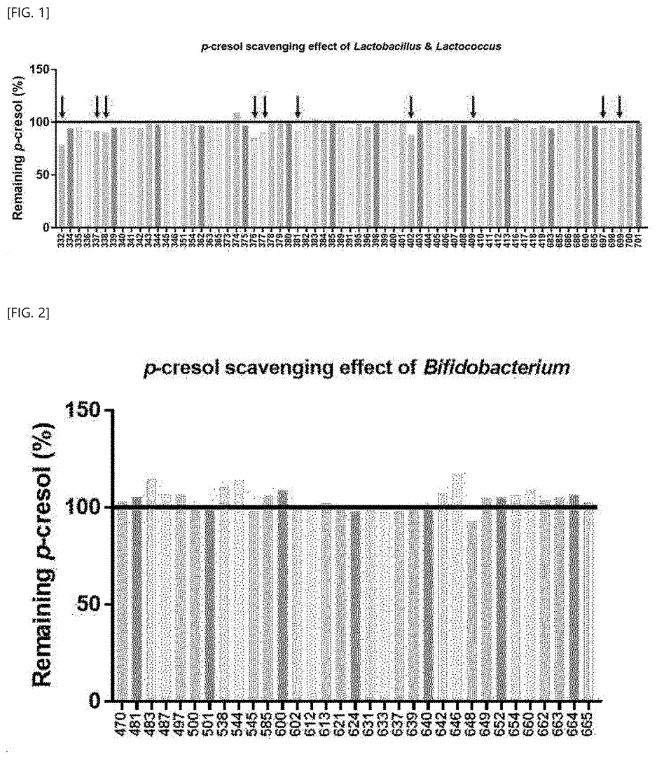

As a result, although most of the Lactobacillus and Lactococcus strains did not have high degrading ability, 10 strains showing some excellent p-cresol degrading ability were first selected (FIG. 1). It was confirmed that most of the Bifidobacterium strains did not degrade p-cresol (FIG. 2).

1-3. Secondary Selection of Strain

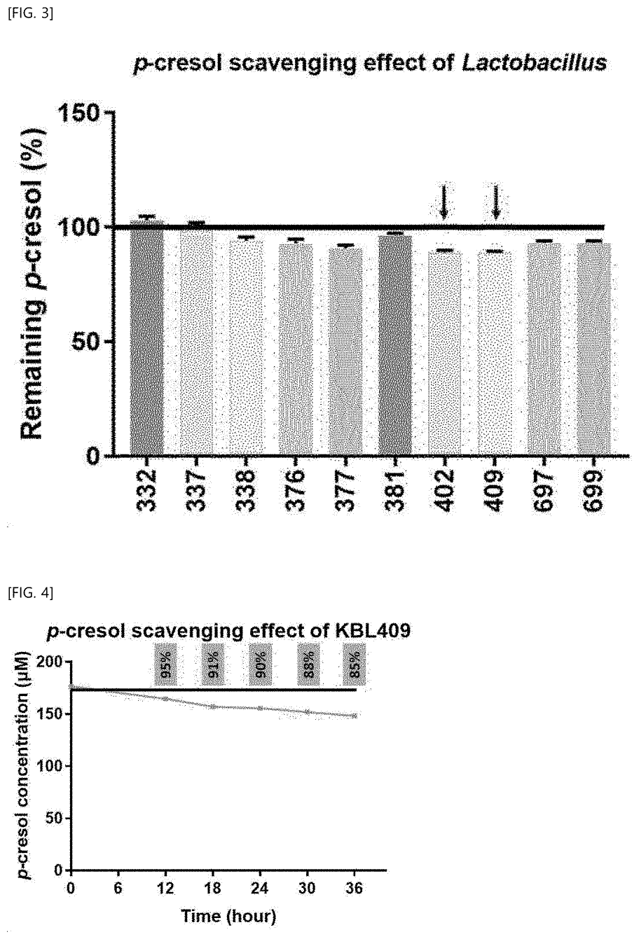

As a result of performing a secondary p-cresol degradation evaluation experiment on 10 Lactobacillus strains whose p-cresol degrading ability was confirmed in Example 1-2, it was confirmed that KBL402 and KBL409 belonging to Lactobacillus acidophilus species had the highest p-cresol degrading ability (FIG. 3). The two strains were judged to be almost similar in the 16S rDNA sequence, and KBL409 strain was selected and further experiments were performed.

1-4. Measurement of the Degrading Ability of p-Cresol of KBL409 Strain

As a result of measuring p-cresol degrading ability over time in the same manner as in Example 1-2 for KBL409, it was confirmed that KBL409 may reduce p-cresol in the culture medium by 95% after 12 hours of treatment and 85% after 36 hours (FIG. 4). As a result, it was found that, in particular, KBL409 exhibited excellent p-cresol degrading ability, effectively alleviating renal disease caused by excessive accumulation of p-cresol.

### Example 2. Confirmation of Anti-Inflammatory Effect of KBL409

Since pyroptosis, one of the important mechanisms of cell death in renal disease, occurs along with the inflammatory response, it is necessary to verify the renal protective effect through the improvement of the inflammatory response. In order to confirm the anti-inflammatory effect as well as the p-cresol degrading ability of KBL409, the expression levels of inflammatory and anti-inflammatory markers were confirmed using peripheral blood mononuclear cells (PBMC).

2-1. Preparation of PBMCs and Processing of Strain

Human-derived PBMCs (Zen-Bio, Inc., Research Triangle Park, N.C., USA) were cultured in RPMI-1640 medium (Gibco, Paisley) containing 1% penicillin/streptomycin, 1% gentamicin and 10% FBS. UK). Cultured PBMCs (2×10⁵ cells) were placed in a 96-well plate and treated with 1 μg/mL of an anti-CD3 antibody that activates T cells (OKT3; Thermo Fisher Scientific, Inc., Waltham, Mass., USA), and then KBL409 strain or Escherichia coli in a ratio of 1:100 was added and cultured at 37° C. for 72 hours.

2-2. Measurement of Inflammatory and Anti-Inflammatory Cytokine

After strain treatment, only the supernatant of cultured PBMC cells was obtained and the amount of each cytokine was measured. BD Cytometric Bead Array (CBA) Human Th1/Th2/Th17 Cytokine Kit (BD Biosciences) was used to measure IL-2, IL-4, IL-10, IFN-γ, and IL-17A, and IL-13 For the measurement, and IL-13 Human enzyme-linked immunosorbent assay (ELISA) Kit (BMS231-3; Thermo Fisher Scientific) was used to measure IL-13.

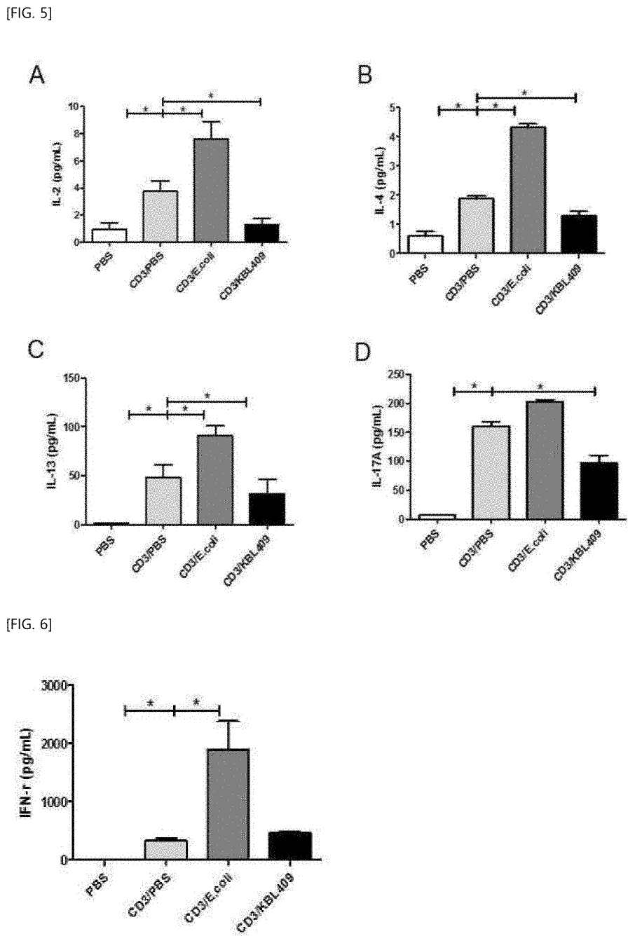

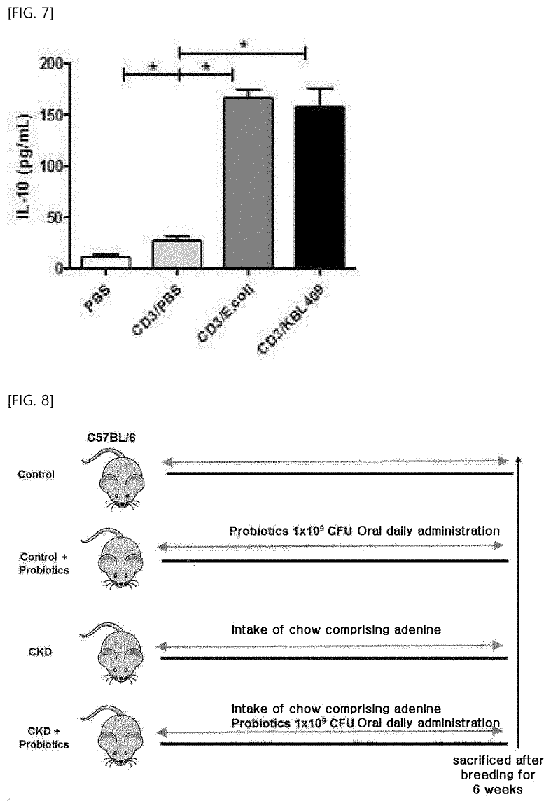

As a result, in the KBL409 treatment group (CD3/KBL409), it was confirmed that Th1 inflammatory cytokines IL-2, Th2 inflammatory cytokines IL-4 and IL-13, and Th17 inflammatory cytokines IL-17A were maintained significantly lower than that of E. coli treatment group (CD3/E. coli) and the PBS treatment group (CD3/PBS) (FIG. 5). On the other hand, IFN-7, another Th1 inflammatory cytokine, was not significantly different from the PBS treatment group (CD3/PBS), but it was confirmed that it was maintained significantly lower than that of the E. coli treatment group (CD3/E. coli) (FIG. 6). In addition, as a result of confirming the expression level of the anti-inflammatory cytokine IL-10 after treatment with KBL409 in PBMC, it was confirmed that the expression of IL-10 in the KBL409 treatment group (CD3/KBL409) was significantly higher that of the PBS treatment group (CD3/PBS) (FIG. 7). Therefore, it was found that KBL409 may suppress the renal and systemic inflammatory responses accompanying chronic renal failure by significantly suppressing the overall expression of inflammatory cytokines in T cells and increasing the expression of the anti-inflammatory cytokine IL-10.

### Example 3. Confirmation of the Effect of KBL409 in Chronic Renal Failure-Induced Mouse Model

A method for inducing renal failure by feeding a feed containing adenine (0.2% adenine) is widely used as a renal failure induction model because it does not require surgery, does not cause death, is relatively easy to take the feed, may be observed for a long period of time, is free from the need for a renalectomy, may observe all renal failure in both kidneys, and may secure sufficient tissue (Jia T et al. A novel model of adenine-induced tubulointerstitial nephropathy in mice. BMC Nephrol 2013; 14:116). In the present invention, using an adenine-induced renal failure model, the effect of improving the change in renal function, renal fibrosis and the like and inflammation upon administration of KBL409 was confirmed. In addition, the effect of reducing uremic toxins in the blood due to the decrease in renal function was also confirmed.

3-1. Model Production of Chronic Renal Failure-Induced Mice and Administration of Strain

In this experiment, 7-week-old C57BL/6 mice with an average body weight of about 20 g were divided into a control group and an experimental group. As shown in FIG. 8, the control group was further divided into two groups, a group administered with the KBL409 strain (Con+KBL409; n=10) and a group not administered (Con; n=10). The experimental group was administered an adenine feed to induce chronic renal failure, and it was divided into a group to which the KBL409 strain was administered (CKD+KBL409; n=10) and a group not administered (CKD; n=10), and the experiment was proceeded with a total of 4 groups of mice. The adenine feed was made to be a diet in which 0.2% of adenine was added to the conventional diet, and 1×10⁹ CFU of the KBL409 strain was orally administered daily. Mice in all groups were sacrificed after breeding for 6 weeks and kidneys were removed (FIG. 8).

3-2. Check for Changes in Renal Function

Blood urea nitrogen (BUN) is a value obtained by measuring nitrogen contained in urea in blood, creatinine is an in vivo filter paper which is a waste product of creatine, a type of protein, and is filtered through the kidney glomeruli, and albuminuria, also referred to as proteinuria, is a mixture of proteins in urine. High albuminuria, BUN, and creatinine content indicate that renal function is impaired. Therefore, in order to confirm the effect of KBL409 on chronic renal failure, the amount of albuminuria, BUN and creatinine was measured using a biochemical analyzer (Automated Chemistry Analyzer, Roche, HITACHI7600) in the urine and blood collected before sacrifice for each mouse group.

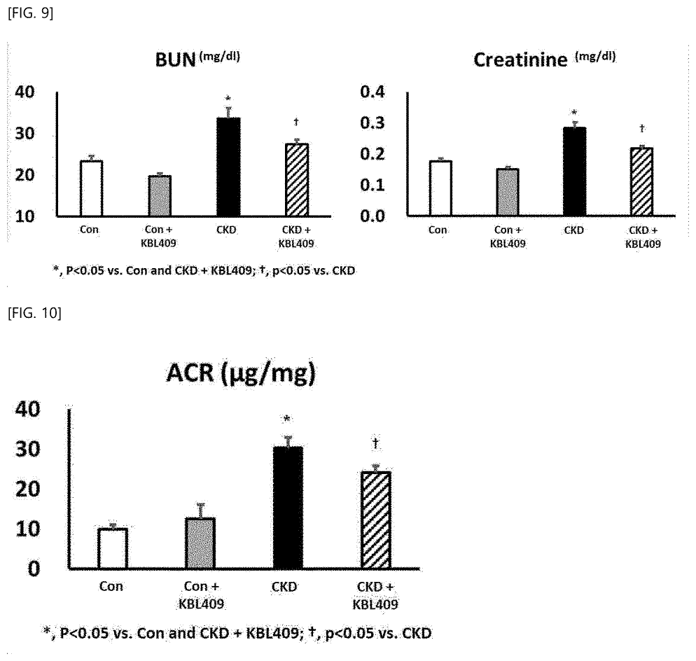

As a result, as may be seen in FIG. 9, the adenine feed-administered group (CKD) showed significant increases in BUN and creatinine concentrations compared to the control group (Con), confirming that chronic renal failure was successfully induced. In addition, the adenine feed and KBL409-administered group (CKD+KBL409) showed significantly lower concentrations of BUN and creatinine compared to the group receiving only the adenine feed (FIG. 9). In the case of albuminuria, the amount of albuminuria was significantly increased in the adenine feed-administered group (CKD) compared to the control group (Con), whereas the adenine feed and KBL409-administered group (CKD+KBL409) significantly decreased the amount of albuminuria compared to that (FIG. 10). Therefore, it was confirmed that the administration of KBL409 of the present invention exhibits an effect of improving renal function by reducing the amount of albuminuria, BUN, and concentration of creatinine, which are representative indicators of renal failure.

3-3. Check for Changes in Kidney Fibrosis

Renal fibrosis refers to a symptom in which renal function is lost due to fibrosis of renal tissue due to various causes such as excessive inflammatory reaction, oxidative stress, and epithelial fibrosis occurring in the renal tissue. It is an important marker of renal disease and is one of the most common symptoms of end-stage renal failure. Accordingly, in order to confirm the effect of KBL409 on renal fibrosis according to chronic renal failure, histopathological examination of renal tissue samples of each group obtained in Example 3-1, expression levels of Procol1a and Acta2 mRNA, and expression levels of Collagen1, Fibronectin, α-SMA and Vimentin were analyzed.

3-3-1. Histopathological Examination

The renal tissue sample obtained in Example 3-1 was prepared as a 4 km-thick tissue section through conventional formalin fixation and paraffin embedded (FFPE). Periodic acid-Schiff (PAS) staining for vascular interstitial expansion and glomerular hypertrophy in renal tissue and Masson's trichrome staining for interstitial fibrosis were performed. After staining, tissue sections of each group were observed under an optical microscope.

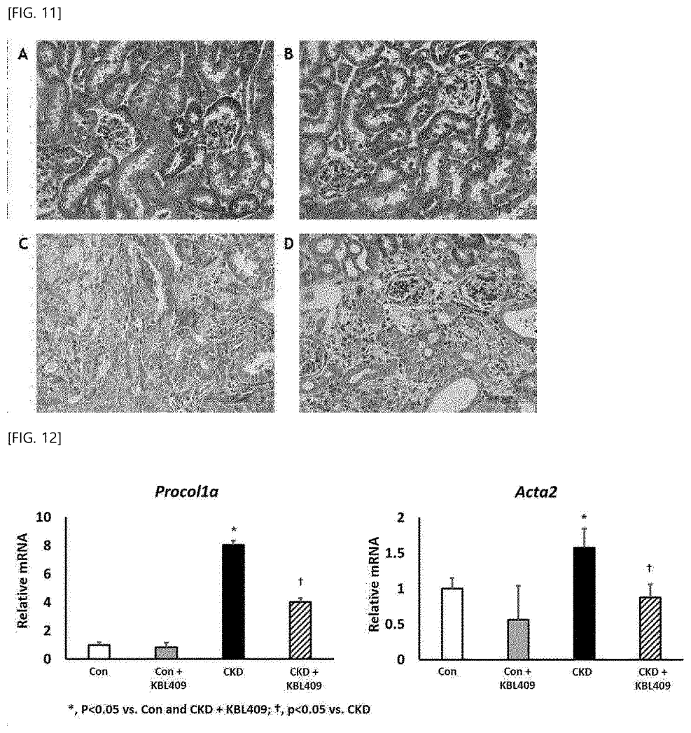

As a result, in the adenine feed-administered group (CKD, FIG. 11C) compared to the control group (Con, FIG. 11A), renal tubular expansion, which is characteristic of renal failure, flattened epithelium, and tubular interstitial expansion and matrix accumulation were observed, and increase in renal fibrosis was observed through Masson's trichrome staining. In the adenine feed and KBL409-administered group (CKD+KBL409, FIG. 11D), the above-mentioned histological changes were clearly observed, and in particular, it was confirmed that fibrosis was significantly reduced.

3-3-2. Identification of Indicators of Renal Fibrosis (1)

In order to determine the degree of expression of the Procol1a and Acta2 genes in the renal tissue, which are indices of fibrosis, mRNA was isolated from the renal tissues of each group obtained in Example 3-1 above, and the mRNA expression levels of Procol 1a and ACta2 gene were analyzed by quantitative polymerase chain reaction (qPCR).

As a result, it was confirmed that the mRNA expression levels of Procol1a and Acta2 genes significantly increased in the adenine feed-administered group (CKD) compared to the control group (Con), whereas a significant decrease was observed in the adenine feed and KBL409-administered group (CKD+KBL409) (FIG. 12).

3-3-3. Identification of Indicators of Renal Fibrosis (2)

The expression of Collagen1, Fibronectin, α-SMA and Vimentin, which are kidney fibrosis-related protein indicators, from the renal tissue of each group obtained in Example 3-1 was confirmed by western blotting.

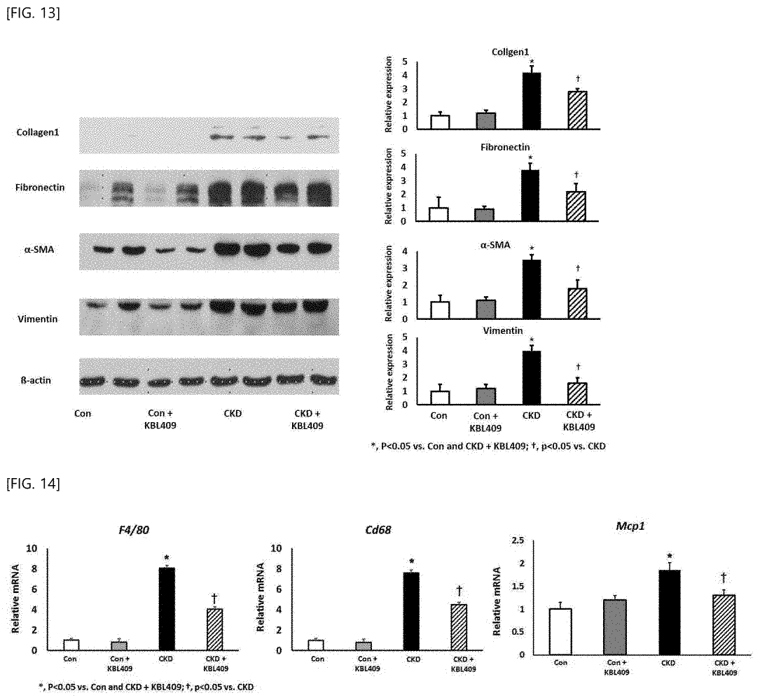

As a result, it was confirmed that the expression levels of Collagen1, Fibronectin, α-SMA, and Vimentin significantly increased in the adenine feed-administered group (CKD), whereas it was significantly decreased in the adenine feed and KBL409-administered group (CKD+KBL409) (FIG. 13).

Therefore, it was found that KBL409 of the present invention is useful for inhibiting the progression of chronic renal failure caused by kidney injury by effectively inhibiting renal fibrosis.

3-4. Check for Changes in Macrophages in the Kidney

The number of F4/80-positive macrophages infiltrating the renal interstitium serves as a marker of renal injury. Accordingly, in order to confirm the effect of KBL409 on the degree of infiltration of macrophages, which is a representative marker of chronic renal failure, changes in macrophages were observed using the renal tissue samples of each group obtained in Example 3-1.

3-4-1. Analysis of F4/80, Cd68 and Mcp1 Expression Level

The expression in the kidney of mRNA of chemokine, Mcp1, on F4/80, Cd68 and monocytes, which are indices of the extent of macrophage infiltration, from the kidney tissues of each group obtained in Example 3-1 was confirmed by quantitative polymerase chain reaction (qPCR) analysis.

As a result, it was confirmed that the mRNA expression levels of F4/80, Cd68 and Mcp1 significantly increased in the adenine feed-administered group (CKD), but significantly decreased in the adenine feed and KBL409-administered group (CKD+KBL409) (FIG. 14).

3-4-3. Histopathological Examination

In the same manner as in Example 3-3-1, after the renal tissue sample obtained in Example 3-1 was prepared into tissue sections with a thickness of 4 μm through conventional formalin fixed paraffin embedded (FFPE), and immunohistochemical staining using antibodies of F4/80 and CD68, which are indicators of the degree of macrophage infiltration, it was observed with an optical microscope.

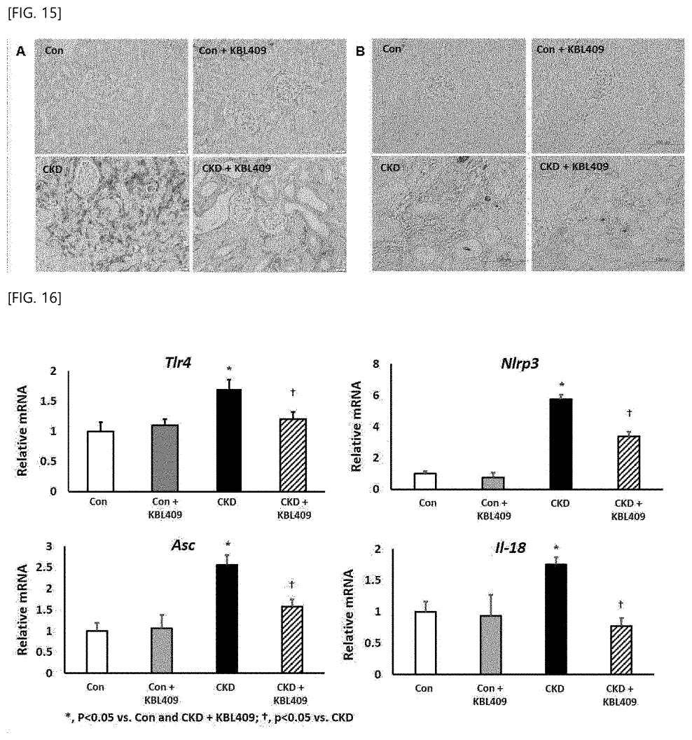

As a result, in the adenine feed-administered group (CKD), the deposition of macrophages in the tubular interstitium was significantly increased compared to the control group (Con), but in the adenine feed and KBL409-administered group (CKD+KBL409), it was confirmed that the degree of infiltration was significantly reduced (FIG. 15).

Therefore, it was found that the administration of KBL409 has the effect of inhibiting the infiltration of macrophages in chronic renal failure.

3-5. Confirmation of Changes in Inflammasome Expression in the Kidney

The inflammasome is a protein complex that recognizes various combinations of inflammation-inducing stimuli and regulates the production of important pro-inflammatory cytokines, such as IL-1β and IL-18, through activation of caspase-1, and pyroptosis, one of the key mechanisms of cell death in chronic renal failure, is known to be caused by inflammasomes. Therefore, in order to confirm the effect of KBL409 on inflammasome expression in the kidney, expression analysis of Tlr4, Asc, Nlrp3, IL-18 and NRLP3 and expression analysis of inflammasome were performed using the renal tissue samples of each group obtained in Example 3-1.

3-5-1. Analysis of Tlr4, Asc, Nlrp3 and IL-18 Expression Levels

From the kidney tissues of each group obtained in Example 3-1, mainly lipopolysaccharide or the like was detected, and mRNA expression of the Tlr4 gene which induces activation of inflammasome, Asc and Nlrp3 which are components of inflamosome, and IL-18 which is a main cytokine of the inflammatory reaction process by inflammosome was confirmed by quantitative polymerase chain reaction (qPCR) analysis.

As a result, it was confirmed that the mRNA expression levels of Tlr4, Asc, Nlrp3 and IL-18 significantly increased in the adenine feed-administered group (CKD) compared to the control group (Con), but significantly decreased in the adenine feed and KBL409-administered group (CKD+KBL409) (FIG. 16). Accordingly, it was found that the administration of KBL409 of the present invention has an effect of inhibiting the expression of Tlr4, Asc, Nlrp3, and IL-18 in the kidney induced by chronic renal failure.

3-5-2. Histopathological Examination

After the renal tissue sample prepared in the same manner as in Example 3-3-1 was prepared into tissue sections with a thickness of 4 μm through conventional formalin fixed paraffin embedded (FFPE), and immunohistochemical staining using antibodies of NRLP3, a component of inflammasome, it was observed with an optical microscope.

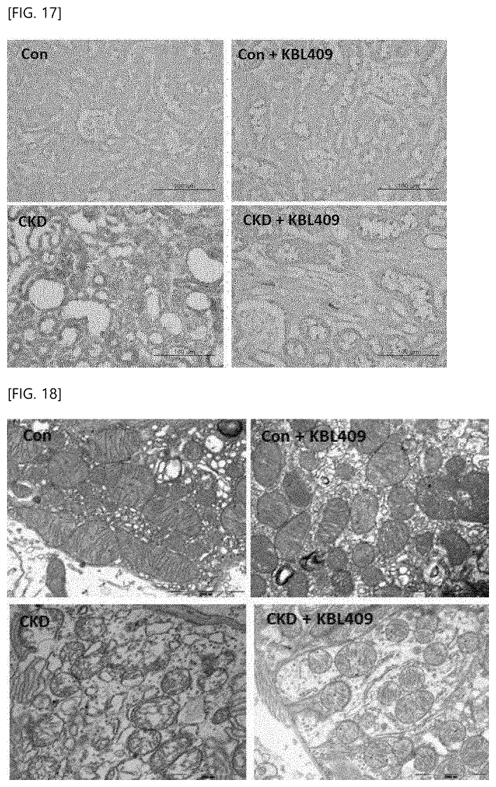

As a result, the expression of NRLP3 in the chronic renal failure-induced renal tissue was significantly increased in the adenine feed-administered group (CKD) compared to the control group (Con), but it was confirmed that it was significantly reduced in the adenine feed and KBL409-administered group (CKD+KBL409) (FIG. 17). Accordingly, it was found that the administration of KBL409 has an effect of inhibiting the expression of NRLP3 increased upon induction of chronic renal failure.

3-6. Check for Changes in Macrophages in the Kidney

One of the important functions of mitochondria is to perform oxidative phosphorylation, which converts energy from fuel metabolites such as glucose or fatty acids into ATP. It is known that such mitochondrial dysfunction is involved in the pathogenesis of renal disease. Accordingly, in order to confirm the effect of KBL409 on the morphological change of mitochondria in the kidney induced by chronic renal failure, the renal tissue samples of each group obtained in Example 3-1 were observed with a transmission electron microscope.

As a result, as may be seen from FIG. 18, in the adenine feed-administered group (CKD), the size of mitochondria in the kidney was reduced and the inner membrane was destroyed, so that it could be confirmed that cristae was lost. On the other hand, in the adenine feed and KBL409-administered group (CKD+KBL409), it was confirmed that the mitochondrial structure was restored (FIG. 18). Therefore, it was found that the administration of KBL409 has the effect of restoring the mitochondrial dysfunction that occurs during the onset of chronic renal failure.

3-7. Changes in Systemic Inflammatory Response

When macrophages and lymphocytes are activated due to chronic inflammation in the kidney, various cytokines such as IL-IβTNF-α are secreted, kidney fibrosis is promoted, and collagen accumulation occurs, which leads to kidney failure. Therefore, in order to confirm the effect of KBL409 on the systemic inflammatory response in a mouse model induced with chronic renal failure, the concentrations of IL-6 and TNF-α, cytokines that induce systemic inflammatory responses were measured by enzyme-linked immunoprecipitation assay (ELISA).

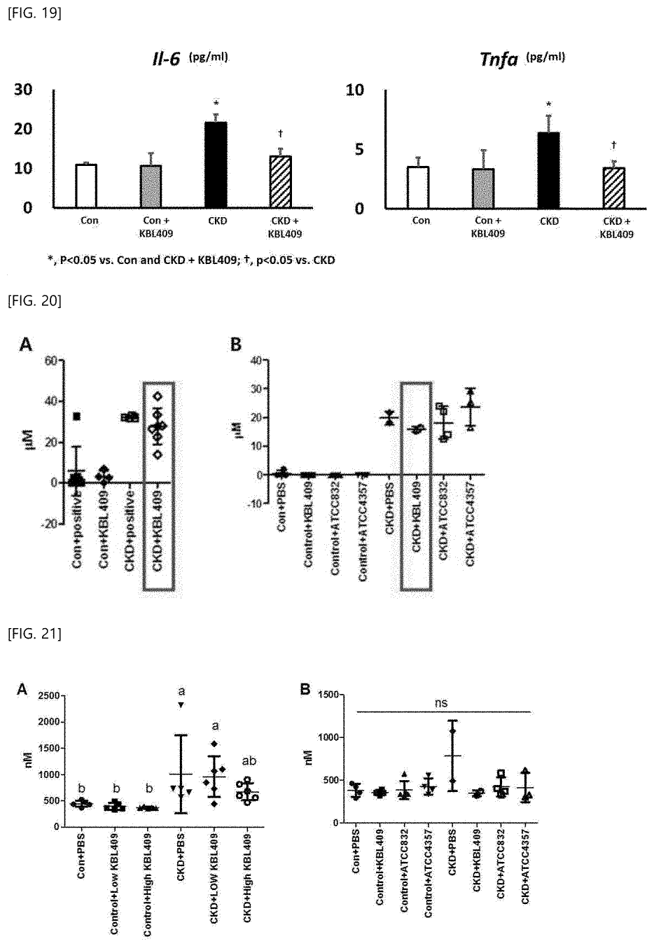

As a result, it was confirmed that the concentrations of both IL-6 and TNF-α were significantly increased in the adenine feed-administered group (CKD) compared to the control group (Con), but significantly decreased in the adenine feed administration group and KBL409-administered group (CKD+KBL409) (FIG. 19). Therefore, it was confirmed that the KBL409 strain was effective in alleviating the systemic inflammatory response by inhibiting IL-6 and TNF-α in a chronic renal failure model.

### Example 4. Comparative Experiment of Changes in the Concentration of Uremic Toxins in Blood

4-1. Comparison of Changes in Blood p-Cresol Concentration

The p-cresol detected in the blood was found to be between 3-300 μM depending on the severity of renal disease, and a calibration curve was derived according to the concentration range. The p-cresol concentration was measured using a blood sample collected before sacrifice. Animal test. Add 2.5 μL of concentrated sulfuric acid to 50 μL of mouse serum, heat at 90° C. for 30 min, add 2.5 μL of the prepared internal standard (0.2 mg/mL 2,6-dimethylphenol, Sigma-Aldrich), and add 50 μL of ethyl acetate as an analysis solvent and mixed with vortex for 1 minute. After centrifugation at 15,000 rpm for 2 minutes, the supernatant was placed in a GC vial (with 250 μL glass insert). GC-MS quantification was performed in the same manner as in the analysis procedure used for strain selection in Example 1.

The p-cresol concentration of each sample was measured based on the calibration curve, and as may be seen from FIG. 20, it was found that in normal mice (Con+PBS), p-cresol was detected at a very low concentration regardless of whether the strain was administered, while the overall p-cresol concentration in CKD-induced mouse samples (CKD+PBS) was increased. When comparing the p-cresol concentration between the E. coli-administered group (CKD+positive) and the KBL409 strain-administered group (CKD+KBL409), it was observed that the p-cresol concentration was measured low in the group administered with the KBL409 strain (FIG. 20A). When the blood concentrations of mice administered with KBL409 strain and other two Lactobacillus acidophilus strains (ATCC832 and ATCC4357) were compared, it was confirmed that the blood p-cresol concentration was reduced when the KBL409 strain was administered (CKD+KBL409) as compared with the p-cresol concentration in the group not administered the strain (CCD+PBS) at the time of inducing chronic renal failure, and that the mice administered the other two types of Lactobacillus acidophilus strains (CDD+ATCC832 and CKD+ATCC4357) had no significant effect on the decrease of the blood p-cresol concentrations: CKD+PBS: 19.8 μM; CKD+KBL409: 15.9 μM; CKD+ATCC832; 18.2 μM; CKD+ATCC4357: 23.7 μM (FIG. 20B). Therefore, it was confirmed that the KBL409 strain of the present invention was significantly superior in terms of the p-cresol reduction effect compared to other Lactobacillus acidophilus strains.

4-2. Comparison of Changes in Blood TMAO Concentration

Blood TMAO was found to be between 1-90 μM depending on the degree of renal disease, and a calibration curve was derived according to the concentration range. The blood TMAO concentration was measured using a blood sample collected before sacrifice. Animal Test. Add 120 μL of ice cold MeOH (LC grade) to 30 μL of mouse serum and vortex for 1 min. After centrifuging at 20,000 g, 4° C. for 20 min, 100 μL of the supernatant was loaded into vivaspin 500, 3KDa, and the filtrate obtained after centrifuging at 15,000 g, 4° C., 30 min was placed in a total recovery vial and used as a sample for TMAO analysis.

Liquid chromatography was used to measure TMAO in serum. UPLC-qTOF was used as the analysis equipment, and analysis was performed in positive ESI ionization and sensitive mode. (A) 0.045% ammonium hydroxide, 0.025% formic acid (pH8.1), and (B) pure acetonitrile were used as mobile phases, at an initial condition of 95% (B), 45% (B) at 2.5 min, 95% B again at 5 min, and 95% B was maintained until 5.5 min and analyzed at a flow rate of 0.4 mL/min. As an analytical column, ACQUITY UPLC BEH Amide Column 130 Å, 1.7 m, 2.1 mm (186004801, waters) was used. As MS parameters, Capillary votage 2 KV, Sampling cone 15, Source offset 10, Source temperature 150° C.Desolvation temperature 200° C.00, Nebuliser 7 were used.

The effects of TMAO concentration and KBL409 in the blood were confirmed in each test group. First, the blood TMAO concentration of the animal test sample administered at high and low concentrations of KBL409 was confirmed. An increase in the TMAO concentration was confirmed upon induction of chronic renal failure (CKD+PBS), and in particular, a marked decrease in the blood concentration of TMAO was confirmed in mice administered with a high concentration of KBL409 (CKD+High KBL409) (FIG. 21A). Next, the concentration of TMAO in the blood was confirmed in chronic renal failure-induced mice administered with three types of L. acidophilus including KBL409. An increase in the TMAO concentration was confirmed due to the induction of chronic renal failure, and the TMAO concentration was decreased in all three L. acidophilus strains administered, but in particular, it was confirmed that the blood TMAO concentration decreased the most in the mice administered with the KBL409 strain (FIG. 21B).

### Example 5. Combined Administration of KBL409 and Additional Probiotic Strain

5-1. Model Production of Chronic Renal Failure-Induced Mice and Administration of Strain

In this experiment, the experiment was carried out by dividing 7 week-old C57BL/6 mice each having an average body weight of 20 g into a total of 6 groups of 10 mice per group, the 6 groups being positive control group (Control), chronic renal failure induction group (CKD), Renadyl™(KIBOW BIOTECH, US) administration positive control group (CKD+positive control), KBL409 alone administered group (CKD+KBL409), KBL409+KBL382 combined administration group (CKD+KBL409+mixed1(382)), KBL409+KBL396 combined administration group (CKD+KBL409+mixed2(396)). The chronic renal failure induction experimental group was administered an adenine feed to induce chronic renal failure, and the adenine feed was made to be a diet in which 0.2% of adenine was added to the normal diet. The KBL409 strain was orally administered with 1×10⁹ CFU daily. In the case of the combination strain (KBL409+KBL382 or KBL409+KBL396), KBL409 was administered at 7×10⁸ CFU and KBL382 or KBL396 was simultaneously administered at 3×10⁸ CFU, and 1×10⁹ CFU was administered daily for the total number of bacteria. Renadyl, a positive control, was also administered orally daily as a composite strain so that the total number of cells was 1×10⁹ CFU. All administration strains were prepared by suspending in PBS containing 0.05% L-cysteine. Mice in all groups were sacrificed after breeding for 6 weeks and kidneys were removed

5-2. Confirmation of Expression Changes in Renal Disease Markers

5-2-1. Analysis of Expression Levels of Nlrp3, Pre-IL18, Ppargc1a, Tfam and Mfn1

From the renal tissue of each group obtained in Example 5-1, mRNA expression level of Nlrp3, a component of the inflammasome complex that has a major effect on the onset of chronic renal failure, and the precursor Pre-IL18 of IL-18, a major cytokine in the inflammatory response process by the inflammasome was analyzed by quantitative polymerase chain reaction (qPCR).

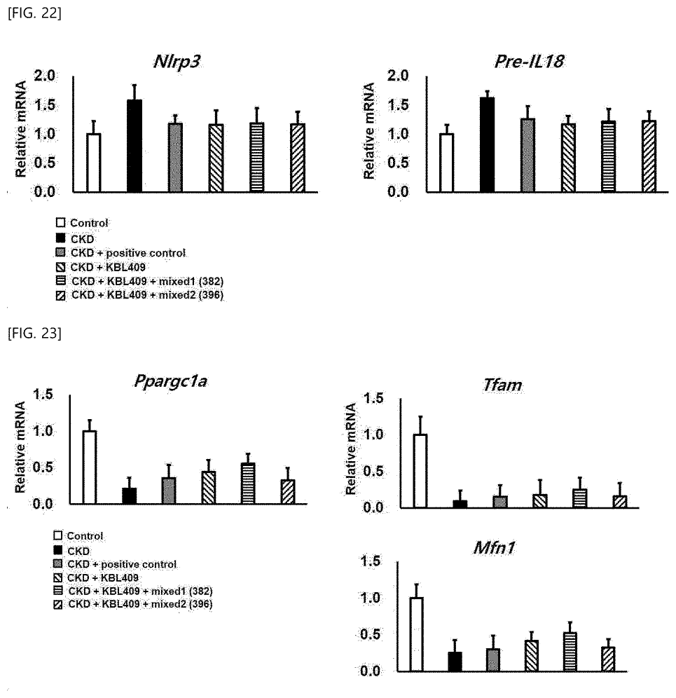

As a result, it was confirmed that the mRNA expression of Nlrp3 and Pre-IL18 increased in the chronic renal failure induction group (CKD) compared to the control group, whereas the expression was reduced in positive control group (CKD+positive control), KBL409 alone administered group (CKD+KBL409), KBL409+KBL382 combined administration group (CKD+KBL409+mixed1(382)), KBL409+KBL396 combined administration group (CKD+KBL409+mixed2(396)) (FIG. 22). Among them, the KBL409+KBL382 combined administration group (CKD+KBL409+mixed1 (382)) showed a superior effect of increasing the expression of Ppargc1a, Tfam and Mfn1 compared to other groups (FIG. 23). Accordingly, the combined administration of KBL409 and KBL382 of the present invention more effectively increases the expression of Ppargc1a, Tfam and Mfn1 in the kidney induced by chronic renal failure model than when the strain is administered alone, and it was found that this synergy may effectively prevent or treat renal disease caused by mitochondrial function injury.

5-2-2. Analysis of Fn and Procol1 Expression Levels and Bax/Bcl2 Ratio

It is known that apoptosis causes ischemic renal dysfunction, and renal ischemia induces apoptosis by increasing Bax/Bcl2 ratio by activating Bax and decreasing Bcl2. That is, in renal epithelial cells, Bax is a pro-apoptotic protein that increases membrane permeability, Bcl-2 is an anti-apoptotic protein that antagonizes “membrane attack” by Bax, and the Bax/Bcl2 ratio is a major determinant of cell death. In this experiment, the mRNA expression levels of Fn (fibronectin) and Procol1, and the expression levels of Bax and Bcl2, which are representative biomarkers of fibrotic diseases, from the renal tissue of each group were analyzed and confirmed by quantitative polymerase chain reaction (qPCR).

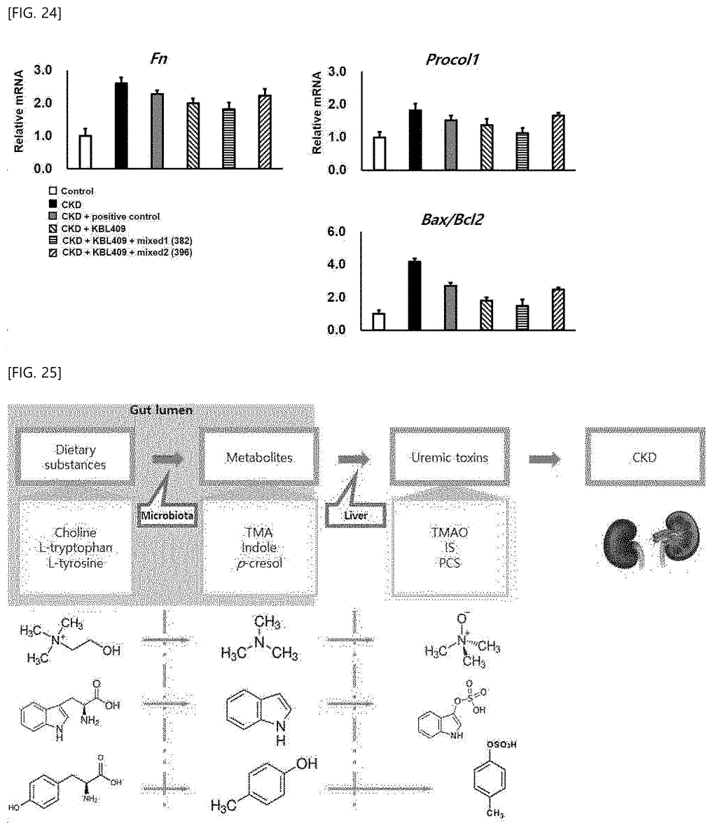

As a result, it was confirmed that the mRNA expression levels of Fn and Procol1 and the Bax/Bcl2 ratio was increased in the chronic renal failure induction group (CKD) compared to the control group, whereas the expression was significantly reduced in positive control group (CKD+positive control), KBL409 alone administered group (CKD+KBL409), KBL409+KBL382 combined administration group (CKD+KBL409+mixed1(382)), KBL409+KBL396 combined administration group (CKD+KBL409+mixed2(396)). Among them, in the KBL409+KBL382 combined administration group (CKD+KBL409+mixed1 (382)), superior Fn, Procol1 and Bax/Bcl2 inhibitory effects were observed compared to other groups (FIG. 24). Thus, it was found that the combined administration of KBL409 and KBL382 of the present invention in an chronic renal failure-induced animal model inhibits the expression of Fn, Procol1 and Bax more effectively than when the strain is administered alone, and increases the expression of Bcl2, thereby effectively preventing or treating renal disease with this synergy.

Having thus described certain portions of the present disclosure in detail, it will be apparent to those skilled in the art that such specific description is only a preferred embodiment and is not intended to limit the scope of the invention. Accordingly, the substantial scope of the present invention will be defined by the appended claims and their equivalents.

Name of depository institution: Korea Research Institute of Bioscience and Biotechnology Deposit number: KCTC13518BP

Deposit Date: 20180427

Name of depository institution: Korea Research Institute of Bioscience and Biotechnology

Deposit number: KCTC13509BP

Deposit Date: 20180417

Name of depository institution: Korea Institute of Biotechnology and Biotechnology

Deposit number: KCTC13278BP

Deposit Date: 20170529

### INDUSTRIAL APPLICABILITY

The Lactobacillus acidophilus KBL409 (deposit number KCTC 13518BP) strain according to the present invention reduces inflammation of the kidney, reduce the concentration of uremic toxins such as blood urea nitrogen, creatinine and p-cresol to protect the kidney, and thus may be usefully utilized for prophylactic and therapeutic applications of renal diseases including improving renal function and chronic renal failure.

### [Sequence List Free Text]

An electronic file is attached.

1. Lactobacillus acidophilus KBL409 strain with deposit number KCTC 13518BP.

2. The strain according to claim 1, wherein the strain has a 16s rDNA sequence of SEQ ID NO: 1.

3. A food composition comprising at least one selected from the group consisting of the strain of claim 1, the culture of the strain, the lysate of the strain and the extract of the strain.

4. The food composition according to claim 3, wherein the food composition improves renal function.

5. The food composition according to claim 4, wherein the improvement in renal function is due to a decrease in renal inflammation, a decrease in blood concentration of uremic toxin, a decrease in proteinuria, a restoration of renal mitochondrial function and/or a decrease in renal fibrosis.

6. An animal feed composition comprising at least one selected from the group consisting of the strain of claim 1, the culture of the strain, the lysate of the strain, and the extract of the strain.

7. A pharmaceutical composition for preventing or treating renal disease, comprising at least one selected from the group consisting of the strain of claim 1, the culture of the strain, the lysate of the strain, and the extract of the strain.

8. The pharmaceutical composition according to claim 7, wherein the prevention or treatment of renal disease is due to a decrease in renal inflammation, a decrease in blood concentration of uremic toxin, a decrease in proteinuria, a restoration of renal mitochondrial function and/or an inhibition of renal fibrosis.

9. The pharmaceutical composition according to claim 8, wherein the uremic toxin is blood urea nitrogen, blood creatinine and/or blood p-cresol.

10. The pharmaceutical composition according to claim 7, wherein the renal disease is selected from a group consisting of uremia, chronic renal failure, acute renal failure, subacute renal failure, renal fibrosis, glomerulonephritis, pyelonephritis, interstitial nephritis, proteinuria, diabetic nephropathy, hypertensive nephropathy, malignant neurosis, lupus nephritis, thrombotic microangiopathy, transplant rejection, glomerulopathy, renal hypertrophy, renal hyperplasia, contrast agent induced nephropathy, toxin induced kidney injury, oxygen free-radical mediated nephropathy, polycystic renal disease and nephritis.

11. The pharmaceutical composition according to claim 7, further comprising at least one selected from the group consisting of an additional probiotic strain, a culture of the strain, a lysate of the strain, and an extract of the strain.

12. The pharmaceutical composition according to claim 11, wherein the additional probiotic strain is at least one selected from Lactobacillus paracasei and Lactobacillus plantarum.

13. The pharmaceutical composition according to claim 12, wherein the Lactobacillus paracasei is Lactobacillus paracasei KBL382 (deposit number KCTC13509BP), and the Lactobacillus plantarum is Lactobacillus plantarum KBL396 (deposit number KCTC13278BP).

14. A method for preventing or treating renal disease, comprising administering to an individual in need thereof at least one selected from the group consisting of the strain of claim 1, a culture of the strain, a lysate of the strain, and an extract of the strain.

15. A use of a composition for preparing a medicament for preventing or treating renal disease, comprising at least one selected from the group consisting of the strain of claim 1, the culture of the strain, the lysate of the strain and the extract of the strain.
